(19) United States
(12) Patent Application Publication
(10) Pub. No.: US 2009/0216108 A1
(43) Pub. Date: Aug. 27, 2009

(54) Real-time magnetic resonance imaging and peripheral arterial tone in sleep apnea diagnosis

(76) Inventors: Jose E. Barrera, San Antonio, TX (US); Andrew B. Holbrook, Mountain View, CA (US); Juan M. Santos, Palo Alto, CA (US); Gerald R. Popelka, Menlo Park, CA (US)

Correspondence Address: LUMEN PATENT FIRM, 2345 YALE STREET, SECOND FLOOR, PALO ALTO, CA, 94306, US

(21) Appl. No.: 12/322,336

(22) Filed: Jan. 30, 2009

Related U.S. Application Data
(60) Provisional application No. 61/063,075, filed on Jan. 30, 2008.

(51) Int. Cl. A61B 5/055; A61B 5/0205

(52) U.S. Cl. 600/410; 600/484

(57) ABSTRACT

Obstructive sleep apnea diagnosis based on peripheral arterial tone (PAT) and real-time magnetic resonance imaging (MRI) is provided. PAT and other physiological data are used to identify respiratory events experienced by a subject with obstructive sleep apnea. MRI images are recorded at least during the respiratory events to identify airway obstructions responsible for the sleep apnea events. The MRI images are recorded in real-time at a high frame rate during the sleep apnea events. The PAT signals and MRI images are collected while the subject is in a sleep state. PAT signals can also be used to identify sleep apnea events and trigger an MRI device to begin recording, thereby reducing the costs associated with recording extraneous and large amounts of MRI data.

### CROSS-REFERENCE TO RELATED APPLICATIONS

This application claims priority from U.S. Provisional Patent Application 61/063,075 filed Jan. 30, 2008, which is incorporated herein by reference.

### FIELD OF THE INVENTION

The invention relates generally to sleep apnea diagnosis. More particularly, the present invention relates to imaging airway obstructions for sleep apnea diagnosis using magnetic resonance imaging and peripheral arterial tone.

### BACKGROUND

Obstructive sleep apnea (OSA) is a sleep disorder that causes sufferers to experience recurrent pauses during sleep, typically due to soft tissue obstructing the airway and preventing normal breathing. Sufferers of OSA experience frequent arousal periods, fragmented sleep, and sleep deprivation. These symptoms can cause drowsiness and weariness during non-sleep periods, which can increase the likelihood for accidents and can decrease productivity. More severely, some studies have found that OSA is related to an increase risk of death from cardiovascular causes. Though sleep apnea is a serious problem, sleep apnea diagnosis faces many difficulties, in part because diagnosis during natural sleep can be challenging and sleep apnea events only last for transient and unpredictable periods of time.

Existing techniques of sleep apnea diagnosis rely on capturing images of obstructions during the sleep apnea events. In particular, magnetic resonance imaging (MRI) has been used to investigate airway obstructions in OSA patients. Since respiratory events occur throughout a sleeping period at unpredictable times, MRI images must be recorded throughout the sleeping period, thereby requiring huge amounts to data to be collected. To identify the obstructions responsible for the sleep apnea events, sleep experts typically must sift through the large amount of data. This process is wasteful in time, can be expensive, and can be error prone as certain sleep apnea events are easily missed.

Electroencephalography (EEG) techniques have been developed to identify sleep apnea events based on their effects on EEG signals. It has been proposed to combine EEG measurements with MRI imaging to identify and image sleep apnea events. However, simultaneous MRI and EEG data collection can be daunting, particularly during natural sleep. In addition, identification of sleep apnea events based on EEG signals is a difficult task that can typically only be accomplished by knowledgeable experts; automation of this task is generally unreliable and complicated. Doctors without EEG expertise would be unable to easily decipher the EEG signals for sleep apnea events, particularly in real-time.

The present invention addresses at least the difficult problems of sleep apnea diagnosis and advances the art with real-time imaging and identification of airway obstructions responsible for sleep apnea events.

### SUMMARY OF THE INVENTION

The present invention is directed to obstructive sleep apnea diagnosis based on peripheral arterial tone (PAT) measurements and real-time magnetic resonance imaging (MRI). A preferred embodiment for obstructive sleep apnea diagnosis includes collecting PAT measurements of a subject while the subject is in a sleep state; identifying one or more respiratory events (REs) experienced by the subject, wherein the REs are identified based on the collected PAT measurements, and wherein a time is associated with each of the REs; imaging the subject with a MRI device to produce one or more MRI images; and correlating the times of the REs with corresponding times of the MRI images to identify the MRI images during the REs. MRI images of REs can be used to identify obstructions, particularly to an airway of the subject, responsible for one of said REs. In a preferred embodiment, the REs are identified in real-time while said RE is occurring.

Preferably, a RE is identified based on a decrease in the PAT measurements, such as a decrease of greater than or equal to about 40% in the PAT measurements. Embodiments of the present invention include collecting other physiological data in addition to the PAT measurements to identify REs. In an embodiment, the pulse rate of the subject is measured and a RE is identified based on an increase in the pulse rate and a decrease in the PAT measurements. In another embodiment, the oxygen hemoglobin saturation amount of the subject is measured and a RE is identified based on a decrease in the oxygen hemoglobin saturation amount and a decrease in the PAT measurements.

Another embodiment includes collecting PAT measurements of a subject in a sleep state; identifying a RE experienced by the subject based on the collected PAT measurements, wherein the RE is identified in real-time while the RE is occurring; triggering a MRI device to image the subject when the RE is identified; and imaging the subject with the MRI device after the MRI device is triggered, wherein the imaging produces one or more MRI images of the subject at least during the RE experienced by the subject.

The present invention is also directed to a system for identifying REs for sleep apnea diagnosis. The system includes an MRI device for imaging a subject in a sleep state to produce one or more MRI images of the subject; a PAT device for collecting one or more PAT measurements of the subject, wherein the PAT device is operable during the MRI imaging; and an identification module for identifying one or more REs, wherein the REs are identified based on the collected PAT measurements, wherein a time is associated with each of the REs, wherein the subject is imaged during at least one of the identified REs, and wherein the MRI images are used to identify an airway obstruction in the subject that caused the RE. In an embodiment, the system also includes a triggering mechanism to trigger the MRI device to produce MRI images, wherein the triggering mechanism triggers the MRI device when one or more of the REs are identified.

In an embodiment, the MRI device produces images at a rate ranging from about 5.5 fps to about 33 fps. Preferably, the REs are identified in real-time while the REs are occurring. The system can also include devices for measuring other physiological data, such as pulse rates and oxygen hemoglobin saturation amounts. In an embodiment, the PAT device is shielded during MRI imaging.

### BRIEF DESCRIPTION OF THE FIGURES

The present invention together with its objectives and advantages will be understood by reading the following description in conjunction with the drawings, in which:

FIG. 1 shows an example of peripheral arterial tone (PAT) and other physiological measurements used to identify a respiratory event (RE) according to the present invention.

FIG. 2 shows an example of a system having a MRI device and a PAT device for sleep apnea diagnosis according to the present invention.

FIG. 3 shows an example of MRI images registered with PAT measurements for imaging REs according to the present invention.

FIG. 4 shows a flow chart of airway obstruction identification and imaging using PAT and MRI according to the present invention.

FIG. 5 shows a flow chart of an example method for triggering MRI recording of REs based on collected PAT measurements according to the present invention.

### DETAILED DESCRIPTION OF THE INVENTION

Real-time sleep apnea diagnosis for obstructive sleep apnea (OSA) subjects can be a daunting task. Sleep apnea diagnosis requires identifying respiratory events (REs) and imaging the airway obstructions during the REs. The task is particularly daunting because the subject to be diagnosed must be in a state of sleep, preferably natural sleep. The present invention is directed to sleep apnea diagnosis using real-time magnetic resonance imaging (RT-MRI) and peripheral arterial tone (PAT) measurements. The present invention includes systems and methods to identify REs while they are occurring and to image obstructions in the subject's airways that are responsible for the REs.

The present invention involves using PAT to identify REs. Respiratory events can include sleep apnea events, airway obstruction events, breathing pauses during sleep, or any other events that are related to sleep apnea. PAT measurements are based on pulsatile volume changes in the arteries, particularly in one or more fingers of a subject. PAT is related to nervous system activity, such as those occurring during REs. Peripheral arterial tone is also referred to as pulse arterial tone, and the two terms are used interchangeably herein.

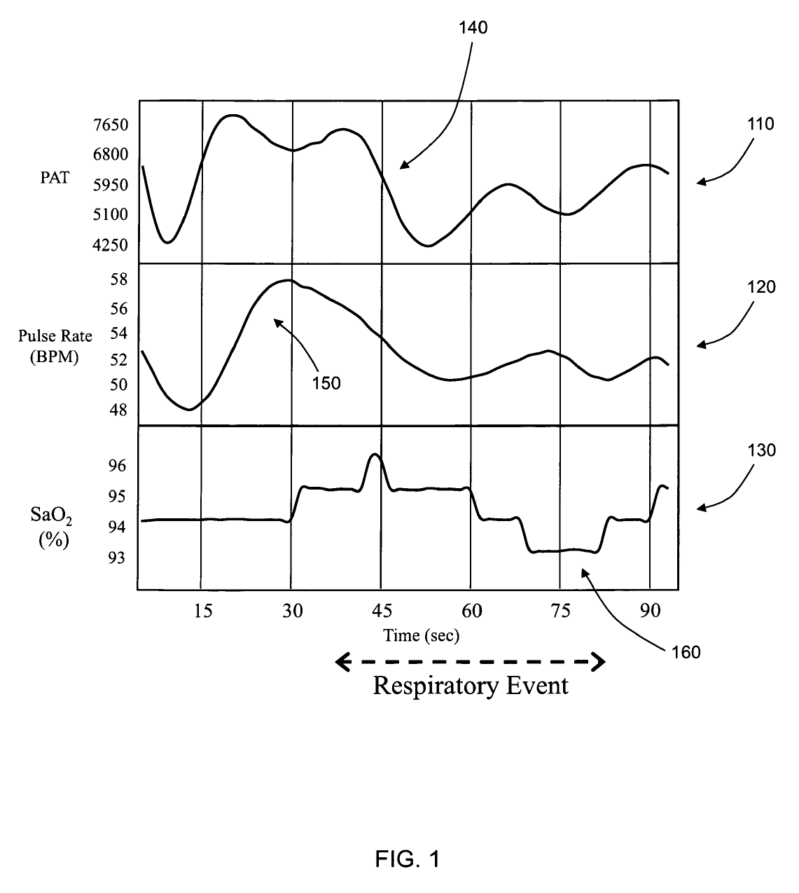

FIG. 1 shows an example plot of PAT measurements 110 versus time for a subject undergoing sleep. In a preferred embodiment, a decrease 140 in the PAT signal 110 indicates that the subject is experiencing a RE. Reduction or attenuation of PAT is referred to as vasoconstriction, the magnitude of which generally determines the severity of a RE. In a particular embodiment, a RE is identified by a decrease of greater than or equal to about 40% in the PAT signal amplitude 110. In FIG. 1, the dashed line shows the duration of a RE.

In a preferred embodiment, REs are identified based on the PAT measurements and one or more other physiological measurements, such as changes to the pulse rate, changes to the oxygen hemoglobin saturation amount (SaO₂), and actigraphy signals for identifying sleep and wake states. FIG. 1 shows pulse rate measurements 120 and SaO₂ measurements 130 in conjunction with the PAT measurements 110. In an embodiment, the subject's pulse rate and PAT signals are measured approximately simultaneously and a drop in the PAT signals 140 associated with an increase in the pulse rate 150 identifies a RE. In another embodiment, a RE is identified by decreases in PAT measurements 140 and SaO₂ percentage 160. It is noted that embodiments of the present invention can rely on any combination of physiological measurements with PAT measurements for identifying REs.

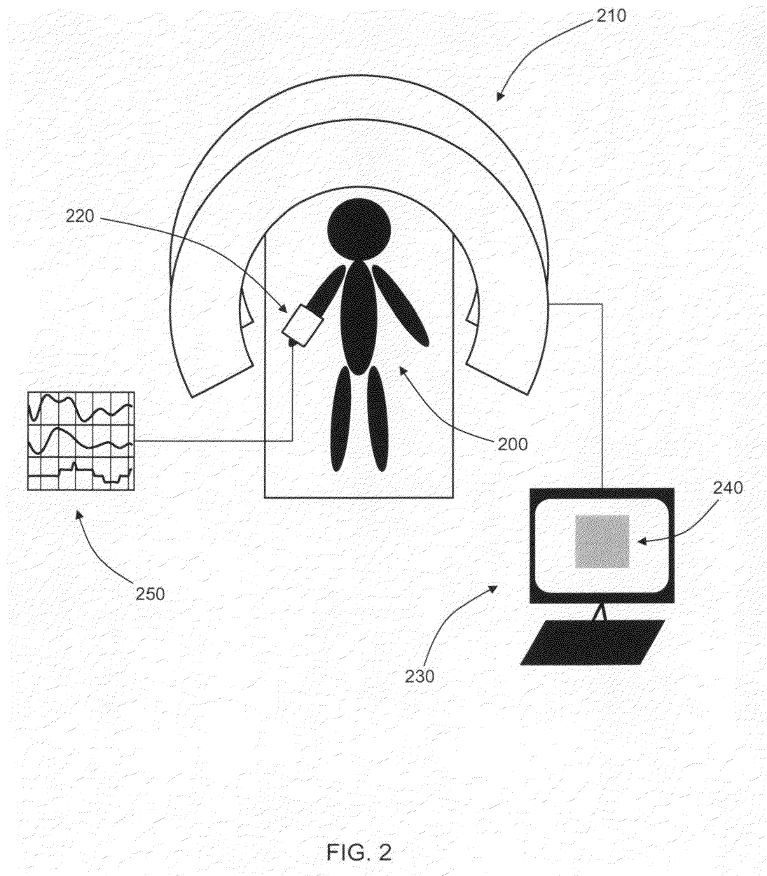

FIG. 2 shows a system embodiment of the present invention for diagnosing sleep apnea experienced by subject 200. The system shown in FIG. 2 includes a MRI device 210 and a PAT device 220 for imaging obstructions and collecting PAT measurements, respectively. In an embodiment, the MRI device 210 includes a receive coil that encircles the face of subject 200 to optimize imaging of the upper airway. The subject 200 is in a sleep state during sleep apnea diagnosis. Preferably, the subject 200 is in a state of natural sleep, i.e. the subject 200 is not experiencing induced sleep, such as with the use of sleep-inducing drugs. Though FIG. 2 shows the subject 200 in a horizontal position, alternative embodiments include an upright or vertical position for the subject 200.

In a preferred embodiment, the MRI device 210 is a real-time MRI device capable of recording MRI images at a high frame rate. Since the locations of obstructions are generally restricted to the upper airway or palate of the subject 200, detailed and high resolution MRI images are not necessary. Since the resolution can be reduced, the MRI device can collect images at a high rate of frames per second (fps). Preferably, the MRI device produces images at a rate ranging from about 5.5 fps to about 33 fps. Effective frame rates can be increased by using a sliding window algorithm, as described by Pauly et al., in U.S. Pat. No. 6,975,751, titled “Method and Apparatus for Construction of Non-Uniformly Sampled Data.” For example, by using the above-referenced sliding window algorithm, a frame rate of 5.5 fps can be effectively increased to 33 fps. In the present context, real-time MRI refers to the collection of MRI images at an adequate frame rate to detect airway obstructions. Real-time can include short time lags and memory buffers for processing and displaying the MRI images.

MRI images collected from MRI device 210 can be stored, processed, or displayed. In FIG. 2, MRI device 210 is communicatively connected to a computer 230 having a monitor for displaying the collected MRI images 240. It is noted that the present invention can include any other tools known in the art for processing, storing, and/or displaying MRI images. The MRI images are used to identify obstructions responsible for REs. In particular, MRI images enable one to observe the positions of the palate, tongue, epiglottis, and posterior pharyngeal wall during a RE.

The embodiment shown in FIG. 2 also includes a PAT device 220 for measuring PAT signals, and optionally, other physiological signals of the subject 200. In a preferred embodiment, the PAT device 220 is a wearable device, particularly on the hand, wrist, or arm of the subject 200. A small wearable PAT device allows the subject 200 to experience a natural sleep state and simulates a realistic sleeping period for the subject 200.

It is important to note that the PAT device 220 collects PAT measurements 250 while the MRI device 210 is operating. In an embodiment, the PAT device 220 is shielded, such as with an aluminum cover, to protect RF noise of the PAT device 220 from being introduced into the collected MRI images. In addition, the high magnetic field environment during the operation of MRI device 210 can cause hazards for the subject 200 wearing a PAT device 220 or anyone else who may be present during the sleep apnea diagnosis. Therefore, in an embodiment, the MRI device 210 preferably operates at a 0.5 Tesla interventional field. This is in contrast to 1.5 Tesla fields used in conventional MRI imaging. However, it is noted that the present invention can be used at any magnetic field strength, including the 1.5 Tesla fields in conventional MRI imaging. In an alternative embodiment, the PAT device 220 comprises a main body and a probe, wherein only the probe is positioned within scanner of the MRI device 210 and the main body is positioned away from regions having substantial magnetic fields.

A preferred embodiment of the present invention includes an identification module for identifying one or more REs experienced by the subject 200. REs are identified based on the PAT measurements as described above and with respect FIG. 1. The identification module can be included with the PAT device, the MRI device, the computer 230, or another device not shown in FIG. 2.

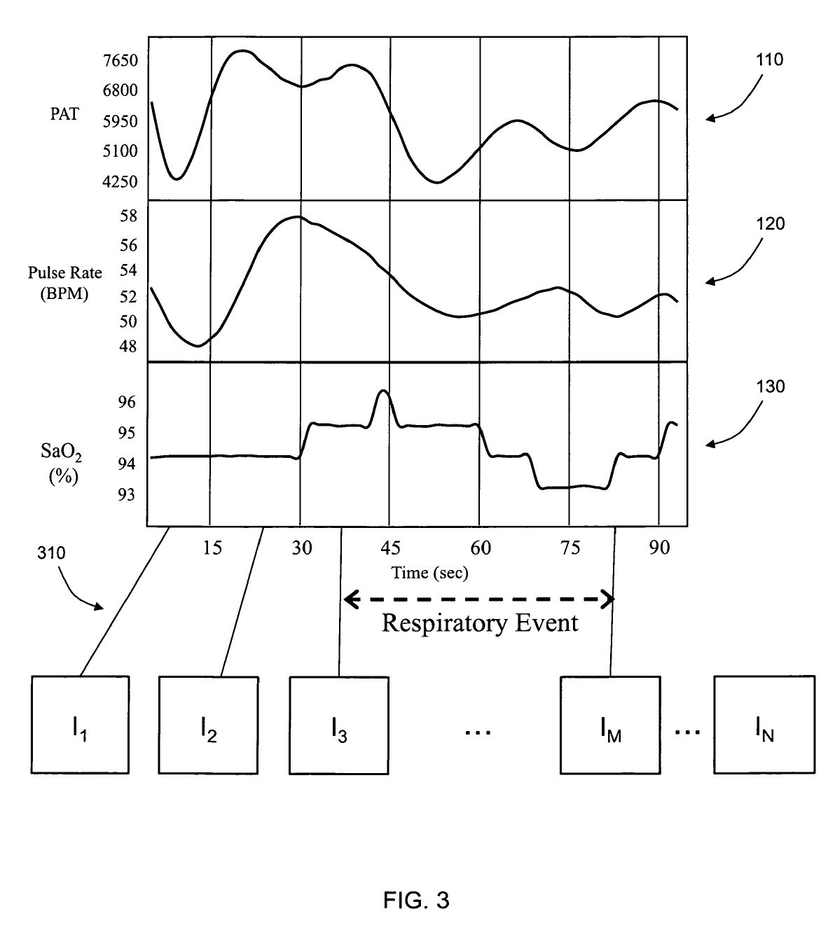

In an embodiment, the PAT device 220 stores and transmits the times of the REs and physiological data collected by the PAT device 220 during the REs. The time associated with each of the REs can be correlated with the times of the MRI images to identify MRI images during a RE. By registering and synchronizing the PAT measurements and the MRI images, the MRI images of interest (i.e. the ones during a REs) can be easily identified and examined for OSA diagnosis, thereby one need not sift through all of the recorded MRI data and only the most pertinent images are viewed. FIG. 3 shows an example of identifying the MRI images associated with a particular RE (denoted by the dashed line). The times associated with the RE shown in FIG. 3 are correlated with the times of a set of MRI images I₁-I_{N}. Since the times of the physiological measurements, including PAT measurements, and the times of the MRI images I₁-I_{N} are registered and synchronized, the MRI images recorded during the RE can be identified as images I₃-I_{M}.

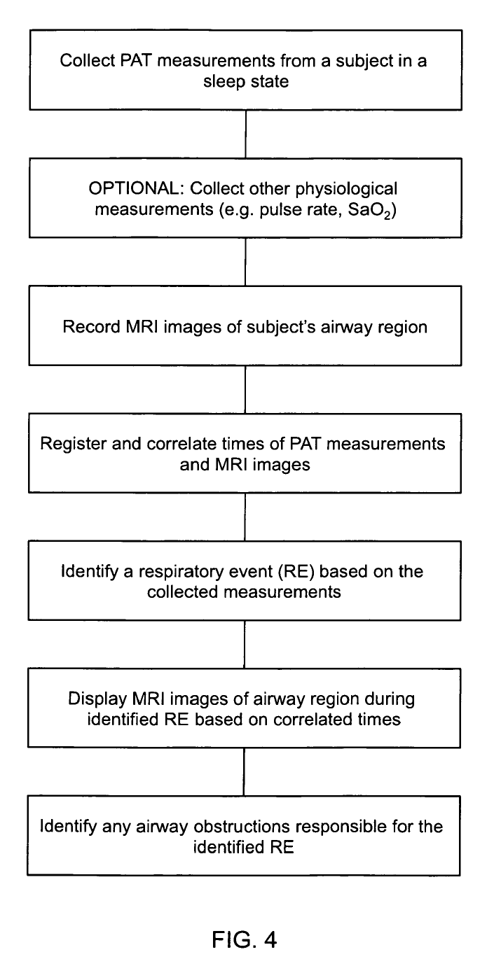

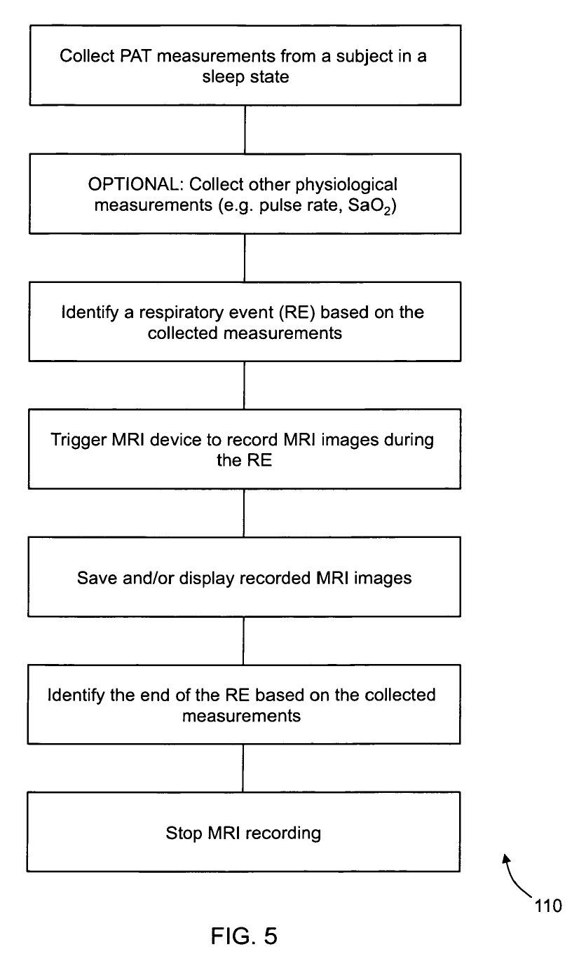

FIG. 4 shows a flow chart of an embodiment of the present invention. It is noted that the embodiment shown in FIG. 4 includes simultaneous recording of MRI images and collecting of PAT measurements, wherein MRI images are recorded even during times when the subject is not experiencing a RE. FIG. 5 shows a flow chart of an alternative embodiment, wherein the amount of MRI data recorded can be further reduced. In the embodiment shown in FIG. 5, a subject is in a sleep state and connected to a PAT device for collecting PAT and other optional measurements. While the subject is asleep and not experiencing any REs, the MRI device does not record any images. Once a RE is identified based on the collected PAT measurements, the MRI device is triggered to record MRI images. MRI images are saved and/or displayed for sleep apnea diagnosis while the subject is experiencing the identified RE. In an embodiment, the cessation of the RE is also identified based on the PAT measurements. At the end of the RE, the MRI device is triggered to stop recording. In an embodiment, the PAT device and the MRI device are communicatively connected to allow the PAT device to trigger the MRI device to start and/or stop recording. By only recording MRI images during a RE, the amount of data required for processing, storage, transfer, and/or display can be greatly reduced.

As one of ordinary skill in the art will appreciate, various changes, substitutions, and alterations could be made or otherwise implemented without departing from the principles of the present invention, e.g. other data processors, displays, and data storage can be included and other physiological data relevant to sleep apnea can be used. Accordingly, the scope of the invention should be determined by the following claims and their legal equivalents.

What is claimed is:

1. A method for obstructive sleep apnea diagnosis, said method comprising:
(a) collecting peripheral arterial tone (PAT) measurements of a subject, wherein said PAT measurements are collected while said subject is in a sleep state;
(b) identifying one or more respiratory events experienced by said subject, wherein said respiratory events are identified based on said collected PAT measurements, and wherein a time is associated with each of said respiratory events;
(c) imaging said subject with a magnetic resonance imaging (MRI) device, wherein said imaging produces one or more MRI images of said subject; and
(d) correlating said times of said respiratory events with corresponding times of said MRI images to identify said MRI images during said respiratory events.

2. The method as set forth in claim 1, wherein one of said respiratory events is identified based on a decrease in said PAT measurements.

3. The method as set forth in claim 2, wherein one of said respiratory event is identified by a decrease of greater than or equal to about 40% in said PAT measurements

4. The method as set forth in claim 1, further comprising measuring a pulse rate of said subject, wherein one of said respiratory events is identified based on an increase in said pulse rate and a decrease in said PAT measurements.

5. The method as set forth in claim 1, further comprising measuring an oxygen hemoglobin saturation amount of said subject, wherein one of said respiratory events is identified based on a decrease in said oxygen hemoglobin saturation amount and a decrease in said PAT measurements.

6. The method as set forth in claim 1, further comprising identifying an obstruction responsible for one of said respiratory events from said MRI images, wherein said obstruction obstructs an airway of said subject.

7. The method as set forth in claim 1, wherein said respiratory events are identified in real-time while said respiratory events are occurring.

8. The method as set forth in claim 1, further comprising registering times of said PAT measurements and said MRI images, wherein said registering is for synchronizing said PAT measurements and said MRI images.

9. A method for obstructive sleep apnea diagnosis, said method comprising:
(a) collecting peripheral arterial tone (PAT) measurements of a subject, wherein said PAT measurements are collected while said subject is in a sleep state;
(b) identifying a respiratory event experienced by said subject, wherein said respiratory event is identified based on said collected PAT measurements, and wherein said respiratory event is identified in real-time while said respiratory event is occurring;
(c) triggering a magnetic resonance imaging (MRI) device to image said subject, wherein said triggering occurs when said respiratory event is identified; and
(d) imaging said subject with said MRI device after said MRI device is triggered, wherein said imaging produces one or more MRI images of said subject at least during said respiratory event.

10. A system for identifying respiratory events for sleep apnea diagnosis, said system comprising:
(a) a magnetic resonance imaging (MRI) device for imaging a subject to produce one or more MRI images of said subject, wherein said MRI images are produced while said subject is in a sleep state;
(b) a peripheral arterial tone (PAT) device for collecting one or more PAT measurements of said subject, wherein said PAT device is operable during said MRI imaging; and
(c) an identification module for identifying one or more respiratory events, wherein said respiratory events are identified based on said collected PAT measurements, and wherein a time is associated with each of said respiratory events,
wherein said subject is imaged during at least one of said identified respiratory events, and wherein said MRI images are used to identify an airway obstruction in said subject that caused said respiratory event.

11. The system as set forth in claim 10, wherein said times of said respiratory events are correlated with corresponding times of said MRI images to identify said MRI images during said respiratory events.

12. The system as set forth in claim 10, further comprising a triggering mechanism to trigger said MRI device to produce said MRI images, wherein said triggering mechanism triggers said MRI device when one or more of said respiratory events is identified.

13. The system as set forth in claim 10, wherein said MRI device produces images at a rate ranging from about 5.5 frames per second to about 33 frames per second.

14. The system as set forth in claim 10, wherein one of said respiratory events is identified based on a decrease in said PAT measurements.

15. The system as set forth in claim 14, wherein one of said respiratory event is identified by a decrease of greater than or equal to about 40% in said PAT measurements

16. The system as set forth in claim 10, further comprising a pulse rate monitor for measuring a pulse rate of said subject, wherein one of said respiratory events is identified based on an increase in said pulse rate and a decrease in said PAT measurements.

17. The system as set forth in claim 10, further comprising an oxygen hemoglobin saturation monitor for measuring an oxygen hemoglobin saturation amount of said subject, wherein one of said respiratory events is identified based on a decrease in said oxygen hemoglobin saturation amount and a decrease in said PAT measurements.

18. The system as set forth in claim 10, wherein said PAT device is shielded during said MRI imaging.

19. The system as set forth in claim 10, wherein said respiratory events are identified in real-time while said respiratory events are occurring.
